(19) United States
(12) Patent Application Publication
(10) Pub. No.: US 2009/0123508 A1
(43) Pub. Date: May 14, 2009

(54) Implantable Drug Depot for Intrathecal Drug Delivery System for Pain Management

(75) Inventors: Ruth CHENG, Natick, MA (US); Toby FREYMAN, Waltham, MA (US)

Correspondence Address: KENYON & KENYON LLP, 1500 K STREET N.W., SUITE 700, WASHINGTON, DC, 20005, US

(73) Assignee: BOSTON SCIENTIFIC SCIMED, INC., Maple Grove, MN (US)

(21) Appl. No.: 12/245,311

(22) Filed: Oct. 3, 2008

Related U.S. Application Data
(60) Provisional application No. 60/977,457, filed on Oct. 4, 2007.

(51) Int. Cl. A61K 9/00; A61K 47/30; A61P 29/00

(52) U.S. Cl. 424/422; 514/772.3

(57) ABSTRACT

The present disclosure provides an intrathecally-implantable depot having a biodegradable core for extended release of pain-relieving drug into the intrathecal space over at time period of at least a month. The present disclosure further provides methods for preparing a self-contained intrathecally-implantable depot and methods for continuously relieving pain in a subject by implanting an intrathecally-implantable depot having a biodegradable core and releasing a therapeutically-effective amount of analgesic composition from the biodegrading core over a time period of at least one month.

### BACKGROUND

Approximately one-fifth of cancer patients are reported to receive inadequate pain relief (Ahmedzai, Current strategies for Pain Control, Annals of Oncology 8 (Suppl.3), 21-24, 1997). Pain control is also important for post-surgical patients. In each of the above contexts, use of analgesic drugs is the generally followed course of treatment for pain relief. An important goal of analgesic therapy is to achieve continuous relief from chronic pain.

Prolongation of the action of the drugs would significantly benefit the patients by continuously maintaining a therapeutic level of pain relief. However, long term pain control is difficult to implement. Currently available forms of analgesics and anesthetics have a relatively limited duration of activity (due primarily to their short plasma half-lives) and some may cause severe toxicity due to their low LD50 values. Consequently, frequent administration is required.

Current methods including continuous delivery of opiates, such as morphine, require complex procedures (e.g., implantation of a pump and catheter line). In addition, such procedures include risks such as pump failure, catheter migration, and infection. Intrathecal drug pumps also require costly and significant maintenance. Infusion is still a restricting mode of application, and the continuous provision of drugs without continuous connection to catheters remains a priority in pain relief treatment.

### SUMMARY

The present disclosure provides an intrathecally-implantable depot having a biodegradable core for extended release of pain-relieving drug into the intrathecal space over at time period of at least a month. The present disclosure also provides an intrathecally-implantable depot having an analgesic composition held in a biodegradable core which releases a therapeutically-effective amount of analgesic in vivo over a time period of at least one month and up to twelve months.

The present disclosure also provides a self-contained depot for long-term intrathecal drug delivery having a biodegradable core containing an analgesic composition provided by a step of encasing said analgesic composition in biodegradable materials that biodegrade at least 50% over a time period of one to 12 months, and forming said biodegradable materials into a biodegradable core comprising a biodegradable casing, a biodegradable matrix, or a combination thereof.

The present disclosure further provides methods for preparing a self-contained intrathecally-implantable depot. A method of continuously relieving pain in a subject is also provided wherein the method includes implanting into said subject an self-contained intrathecally-implantable depot comprising a biodegradable core, and analgesic composition held in the biodegradable core; biodegrading said biodegradable core in vivo over a time period of at least one month; and releasing a therapeutically-effective amount of analgesic composition from the biodegrading core over a time period of at least one month.

### DESCRIPTION OF THE DRAWINGS

In the drawings, wherein like numerals designate like parts throughout the same.

FIG. 1 is a schematic representation depicting an example of an intrathecally-implantable depot of the present disclosure as implanted in a human spine.

FIG. 2 is a schematic representation depicting greatly enlarged views of an example of an intrathecally-implantable depot of the present disclosure.

FIG. 3 is a longitudinal sectional representation of another example of an intrathecally-implantable depot of the present disclosure.

FIG. 4 is a longitudinal sectional representation of a further example of an intrathecally-implantable depot of the present disclosure.

FIG. 5 is a schematic representation a yet further example of an intrathecally-implantable depot of the present disclosure.

FIG. 6 is a schematic representation an alternative example of an intrathecally-implantable depot of the present disclosure.

FIGS. 7-10 are cross-sectional representations of an intrathecally-implantable depot of the present disclosure.

### DESCRIPTION

Various embodiments of the present disclosure will be described in detail with reference to the drawings, wherein like reference numerals represent like parts throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

For the following defined terms, these definitions shall be applied, unless a different definition is given in the claims or elsewhere in this specification.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used herein, “extended release” refers to a pharmaceutical form of release of an active substance and its subsequent absorption are prolonged in comparison with a conventional non-modified form. Typically, “extended release” of an active substance in a patient produces a spread of blood/plasma concentration (at therapeutically effective levels) as a function of time, with an increased apparent elimination half-life and/or reduction in peaks or achievement of a “plateau”. Generally, “extended release” refers to in vivo release and absorption in a patient, but can be modeled in vitro. “Extended release” encompasses the above defined general mode of release, and also encompasses other terms referring to such release kinetics, such as: slow, gradual, prolonged, continuous, controlled, delayed, retard, sustained, etc.

As used herein, “intrathecal” refers to the intrathecal space of a vertebrate spine, typically a mammalian spine, and often a human spine. The intrathecal space is defined as the space within the sheath of dura matter surrounding the spinal cord. The intrathecal space is occupied by cerebrospinal fluid. “Intrathecally-implantable” refers to properties suitable for implantation into the intrathecal space of a spine, including size to fit within an intrathecal space and materials compatible with occupancy of the intrathecal space in the spine of a living patent. Typically, “intrathecally-implantable” refers to size and shape suitable for occupancy of the intrathecal space of a patient's spine without blocking flow cerebrospinal fluid (CSF flow) within the space.

### Intrathecally-Implantable Depot

The present disclosure provides an intrathecally-implantable depot, having a biodegradable core for extended release of pain-relieving drug into the intrathecal space of a spinal cord. The intrathecally-implantable depot, when implanted in the intrathecal space of a patient's spinal cord provides for long-term management of chronic pain. Chronic pain management is achieved by extended release of pain-relieving drug, e.g., analgesic, within the intrathecal space of the spine. Management of chronic pain via implantation of an intrathecally-implantable depot disclosed herein is useful for treatment of any condition including chronic pain enduring of a month or more. Example conditions including chronic pain which may be treated by implantation of a intrathecally-implantable depot described herein include, cancer pain, such as pain related to metastatic cancer, pain related to tumors compressing the spinal nerves, and/or pain related to scarring from previous radiation therapy; failed back surgery syndrome; reflex sympathetic dystrophy; causalgia, arachnoiditis; and chronic pancreatitis.

The intrathecally-implantable depot of the present disclosure is sized for fit within the intrathecal space, for example, of a human spine. Typically, the size and shape of the intrathecally-implantable depot is selected to prevent blockage of cerebrospinal fluid (CSF flow). Often, the intrathecally-implantable depot is rod-shaped. Often, an intrathecally-implantable depot of the present disclosure is flexible. Flexible generally refers to the property of an intrathecally-implantable depot to bend or deform within the confines of an intrathecal space. For example, during movement of the spine. Flexible also refers to the property of an intrathecally-implantable depot to bend or deform during implantation into an intrathecal space, for example for administration through a needle or catheter.

The pain-relieving drug is disposed within the biodegradable core configured for extended release. The biodegradable core and pain-relieving drug are together configured for release of the pain-relieving drug at therapeutically-effective concentration over at time release period of at least a month. Typically, biodegradable core and pain-relieving drug are together configured for release of therapeutically effective amounts of the pain-relieving drug over a time period from one to twelve months. In some instances, the biodegradable core is configured to additionally provides short-term release of one pain-relieving drug as well as being configured the long-term release of therapeutically effective amounts of other pain-relieving drug over a time period longer than one month.

Biodegradable core and pain-relieving drug are generally configured for to have a time release period of at least one month by a combination of structure and selection of biodegradable materials to control biodegradation of the biodegradable core and diffusion of the pain-relieving drug. Biodegradable cores of the present disclosure include at least one biodegradable casing or biodegradable matrix or combinations thereof. Biodegradable materials are selected for the biodegradable cores, for example, biodegradable casing or matrix portions thereof, dependent on the desired drug release profile of the pain-relieving drug into the intrathecal space. Generally, biodegradable materials are selected for extended release of therapeutically effective amounts of the pain-relieving drug over a time period of at least one month. Often biodegradable materials are selected for extended release of therapeutically effective amounts of the pain-relieving drug over a time period from one to twelve months. In some instances, biodegradable materials are selected for extended release of therapeutically effective amounts of the pain-relieving drug over a time period from one to twelve months and to additionally deliver an additional amount of pain-relieving drug over a short-term time period of less than one month.

The intrathecally-implantable depot is generally placed within the intrathecal space via a delivery device. Typically, the delivery device is a low gauge needle, catheter, or other medical device manipulable by medical personnel for accessing the spine of a patient. The delivery device is removed from the intrathecal space after implantation of the intrathecally-implantable depot. In some instances, a medical device, such as a catheter or other surgical intervention may be used to remove the remaining intrathecally-implantable depot from the intrathecal space after desired drug delivery has been achieved. The intrathecally-implantable depot of the present disclosure is self-contained. After implantation in the patient, the intrathecally-implantable depot has no connection outside the body. For example, the intrathecally-implantable depot does not require a catheter connection or a pump. There is no connection extending from the depot outside the intrathecal space or outside the body during long-term drug delivery.

### II. EXAMPLE CONFIGURATIONS OF INTRATHECALLY-IMPLANTABLE DEPOT

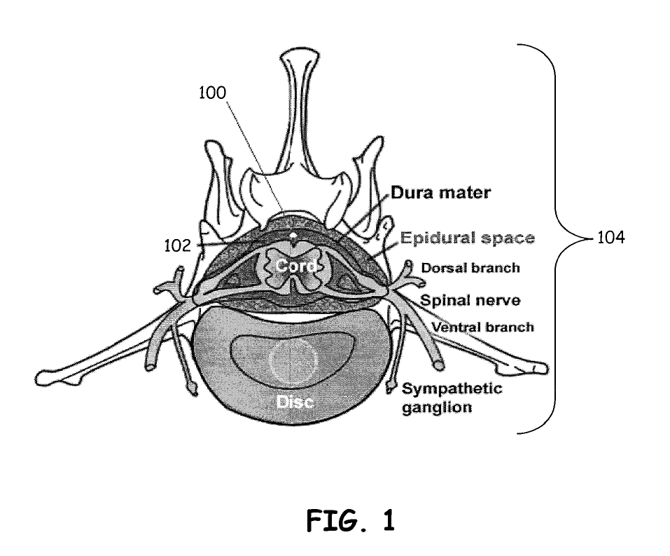

Further description of the intrathecally-implantable depot of the present disclosure is presented in relationship to an intrathecally-implantable depot for purposes of illustration rather than limitation. Referring now to the drawings, FIG. 1 shows a cross-sectional representation of an intrathecally-implantable depot 100, implanted in the intrathecal space 102 of a spinal column 104.

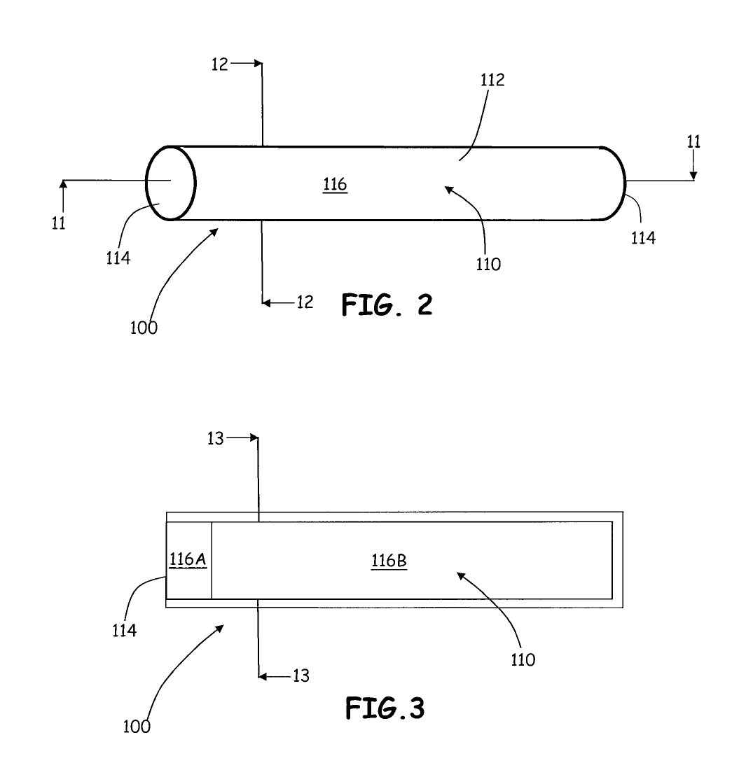

One example of an intrathecally-implantable depot 100 of the present disclosure is shown in FIG. 2. Intrathecally-implantable depot 100 is at least partially constructed of biodegradable core 110. Biodegradable core 110 has an outer surface 112 and two ends 114. Generally, biodegradable core 110 includes one or more biodegradable matrices or biodegradable casings, containing one or more, same or different, pain-relieving drugs dispersed therein. In one possible embodiment of an intrathecally-implantable depot 100 as represented by FIG. 2, biodegradable core 110 consists of biodegradable matrix 116. Frequently, biodegradable matrix 116 is a homogeneous biodegradable polymer having pain-relieving drug dispersed within. Biodegradable matrix 116 is typically solid or alternatively semi-solid having a gel-like consistency. On occasion, outer surface 112 of the biodegradable core 110 may also be the outer surface of depot 100.

In various embodiments of intrathecally-implantable depot 100, depot 100 is flexible, for example being of sufficient flexibility to move, bend or deform in a range of movement that is at least that of the range of movement of a spine, for example a human spine. In general, flexibility is imparted to intrathecally-implantable depot 100 by one or more portions thereof being formed of flexible materials. In various embodiments, biodegradable core 110 is at least partially formed of flexible materials. In various further embodiments, one or more biodegradable matrices or biodegradable casings are formed of flexible materials. On occasion, biodegradable matrix 116 is formed of flexible material.

In alternative embodiments of intrathecally-implantable depot 100 of the present disclosure, biodegradable core 110 includes one or more additional, same or different biodegradable matrices and/or biodegradable casings, containing one or more, same or different, pain-relieving drugs dispersed therein. In addition, intrathecally-implantable depot 100 may include one or more casings which covers or coats, either partially or fully, surface 112 and ends 114 of biodegradable core 110.

FIG. 3 presents a length-wise sectional representation of another example intrathecally-implantable depot 100. Intrathecally-implantable depot 100 of FIG. 3 includes a casing 118 non-continuously surrounding biodegradable core 110. As shown in FIG. 3, casing 118 may be open on one end 114 of intrathecally-implantable depot 110, or alternatively, casing 118 may be open on both ends 114, or have one or more openings within the region covering outer surface 112. In another alternative, casing 118 is impermeable except at one or both ends 114 wherein the casing 118 is permeable to drug diffusion. In one such alternative, one or both ends 114 include a membrane having permeability for drug diffusion. In a further alternative, the membrane has selective permeability for drug diffusion out of the depot, but is impermeable to diffusion of other depot components, for example biodegradable polymers.

In various embodiments, intrathecally-implantable depot 100 includes casing 118 to hold or encase biodegradable core 110 including, but not limited to solid

Casing 118 may be biodegradable, may have delayed degradability in vivo, or may be non-biodegradable. Alternatively, casing 118 is non-biodegradable. Some embodiments of depot 100 having non-biodegradable casing 118 are refillable. requiring subsequent surgical removal. In embodiments, wherein casing 118 is non-biodegradable or has delayed degradability in vivo, casing 118 must be non-continuous in order for the biodegradable core 110 to be in contact with the cerebrospinal fluid upon implantation in the intrathecal space 102.

In alternative examples, casing 118 may fully surround biodegradable depot 100. In such cases, casing 118 is generally biodegradable.

In the example depot 100 of FIG. 3, biodegradable core 110 includes two biodegradable matrices 116A and 116B. In examples including multiple biodegradable matrices, each biodegradable matrix 116 may be fabricated from same or different biodegradable polymer. In addition, each biodegradable matrix 116 may have the same or different extended-release forms (e.g., same or different biodegradability and/or drug release profiles). Also, each biodegradable matrix may incorporate the same or different pain-relieving drug in a similar or different amount. On occasion, biodegradable matrix 116A may be of a form to provide a burst or other short-term duration release of a pain-relieving drug. Typically, such a burst or short-term duration release begins immediately or soon after implantation of the intrathecally-implantable depot 100. On occasions when biodegradable matrix 116A provides short-term duration release, biodegradable matrix 116B provides extended release of the same or different pain-relieving drug over a longer time period of one month or more. In one example embodiment of the intrathecally-implantable depot of FIG. 3, biodegradable matrix provides long-term duration release of 116A provides short-term release of a first pain-relieving drug and biodegradable matrix 116B provides long-term release of a second pain-relieving drug over a time period of, for example, at least one month and up to six months, or even at least one month and up to twelve months.

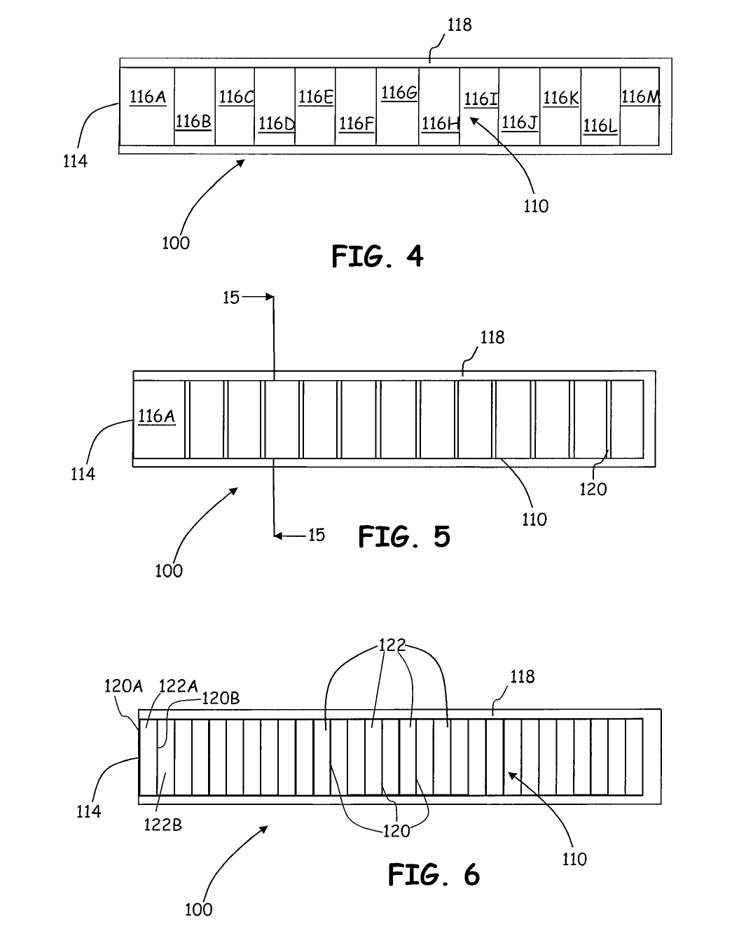

Another example of intrathecally-implantable depot 100 is depicted by length-wise sectional representation of FIG. 4. As shown in FIG. 4, a biodegradable core 110 may include a plurality of biodegradable matrices. As shown in FIG. 4, biodegradable core 110 is divided into a plurality of cross-sections, wherein each cross-section is a biodegradable matrix 116A-M.

FIG. 5 depicts a further example of intrathecally-implantable depot 100. A biodegradable core 110 may include a plurality of biodegradable matrices 116 and additionally comprise one or more biodegradable casings 120 surrounding or separating biodegradable matrices 116. As shown in FIG. 5, biodegradable core 110 is divided into a plurality of cross-sections, wherein each cross-section is a biodegradable matrix 116A-M. In addition each biodegradable matrix is separated, or alternative surrounded, by a biodegradable casing 120.

In an alternative example, intrathecally-implantable depot 100 includes a biodegradable core 110 containing a plurality of units where each unit is surrounded by a biodegradable casing 120. In embodiments of intrathecally-implantable depot including a plurality of units, the plurality includes at least 2 units. A plurality of units is often in a range of 2-100 units inclusive, but on occasion includes 2-4 units inclusive, or 5-20 units inclusive, or 21-100 units inclusive, or in excess of 100 units. Frequently, a biodegradable core 110 including a plurality of units 122 is held together by a casing 118.

The biodegradable casings 120 provide extended release of the pain-relieving drug contained therein. For example, as provided in FIG. 6, casing 118 has one opening at end 114 to allow access of cerebrospinal fluid (not shown) to the encased units 122. As the first biodegradable casing 120A degrades thereby mediating release of the drug within first unit 122A, the next biodegradable casing 120B will become exposed and available for degradation. Generally, the rate of degradation of each biodegradable casing 120 is coordinated with the amount of pain-relieving drug in each unit 122. Typically, the coordination provides for the next unit to begin release when the concentration of drug in the cerebrospinal fluid from the previously-released unit can no longer sustain the desired therapeutically effective concentration.

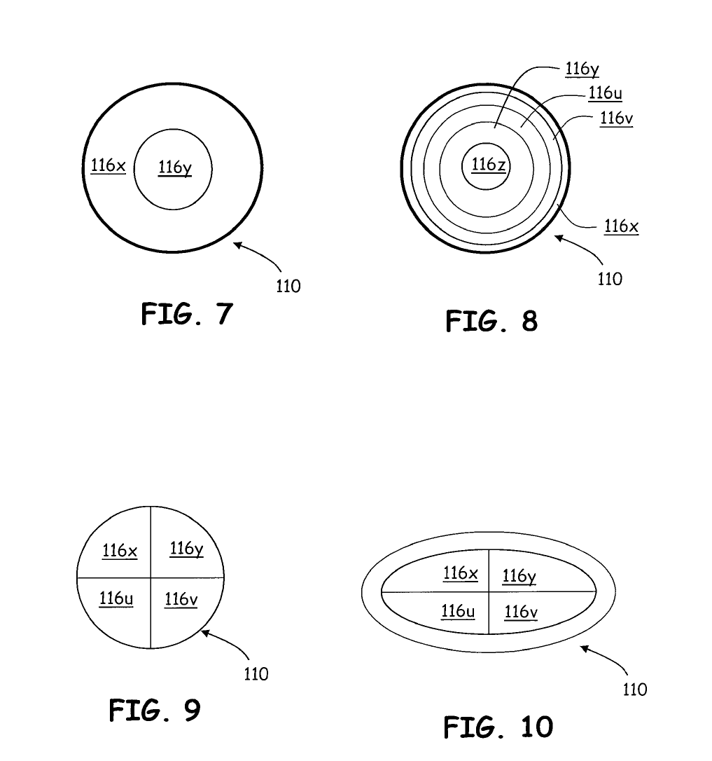

Intrathecally-implantable depot 100 may include biodegradable cores 110 having various cross-sectional configurations of biodegradable matrices and biodegradable casings. FIGS. 7-10 present possible cross-sections of biodegradable core 110, for example as may be seen along line 12-12 of FIG. 2, line 13-13 of FIG. 3, line 14-14 of FIG. 4, and line 15-15 of FIG. 5. A cross-section of biodegradable core 110 may contain one or more biodegradable matrices or biodegradable casings. For example, FIG. 7 depicts a cross-section of a biodegradable core 110 which has two concentrically-layered biodegradable matrices 116x, 116y. In other embodiments, such as depicted in FIG. 8, biodegradable matrix 116 may be formed of a plurality of layers of the same or different biodegradable polymer, containing the same or different pain-relieving drug, in same or different amounts. Alternatively, biodegradable core 110 may be divided longitudinally, for example as depicted in the cross-sectional representation of FIG. 9. In FIG. 9, biodegradable core 110 is divided longitudinally into four quadrants of biodegradable matrix, 116u, 116v, 116x, 116y. Biodegradable matrices 116u, 116v, 116x, 116y may be formed of a plurality of layers of the same or different biodegradable polymer, have same or different drug release profiles, and contain the same or different pain-relieving drug dispersed therein, in same or different quantities.

In various further embodiments, one or more biodegradable matrices 116 or units 120 of the biodegradable cores 110 presented in FIGS. 2-6, may have cross-sections as represented in FIGS. 7-10.

### Pain-Relieving Drug

Pain-relieving drug, as used herein, refers to one or more compounds, or pharmaceutical compositions thereof, which act centrally in a patient to elevate the “pain threshold” (point at which pain is perceived) without disturbing consciousness or altering other sensory modalities. Pain-relieving drug which reduce the perception of pain without a loss of consciousness include analgesics. Analgesics include various classes of compounds including, but not limited to, narcotic analgesics (opiates and opioids), steroid analgesics, and non-steroidal anti-inflammatory drugs (NSAIDS).

Generally, suitable opioids are derivatives of one of five chemical groups: phenanthrenes, phenylheptylamines, phenypiperidines, morphinans, and benzomorphans. Opioids include natural opioids, i.e. opiates, semi-synthetic opioids, fully synthetic opioids, and endogenous opioid peptides. Suitable opioids include morphine, morphine analogs and morphine derivatives, such as, codeine, heterocodeine, morphinone, dihydromorphine, dihydrocodeine, dihydromorphinone, dihydrocodeinone, 6-desoxymorphine, heroin, oxymorphone, oxycodone, 6-methylene-dihydromorphine, hydrocodone, hydromorphone, metopon, apomorphine, normorphine, N-(2-phenylethyl)-normorphine, and oripavine derivatives such as etorphine and buprenorphine.

Example compounds include 4,5-epoxy-methylmorphinan opioid alkaloids (morphine, codeine, and thebaine) and derivatives thereof, including epoxymorphinans (nalorphine, nalbuphine), morphinans (levorphanol), benzomorphans (pentazocine), phenyl-piperidines (pethidine), the 4-anilino-piperidines (fentanyl, Alfentanil, Sufentanil, Remifentanil) and meperidine.

Non-steroidal anti-inflammatory drugs (NSAIDS) include: Salicylates, such as, aspirin, amoxiprin, benorilate, choline magnesium salicylate, diflunisal, faislamine, methyl salicylate, magnesium salicylate, salicyl salicylate (salsalate); Arylalkanoic acids, such as, diclofenac, aceclofenac, acemetacin, bromfenac, etodolac, indometacin, nabumetone, sulindac, tolmetin; 2-Arylpropionic acids (profens), such as, ibuprofen, carprofen, fenbufen, fenoprofen, flurbiprofen, ketoprofen, ketorolac, loxoprofen, naproxen, oxaprozin, tiaprofenic acid, suprofen; N-Arylanthranilic acids (fenamic acids), such as, mefenamic acid, meclofenamic acid; Pyrazolidine derivatives, such as, phenylbutazone, azapropazone, metamizole, oxyphenbutazone, sulfinprazone; Oxicams, such as, piroxicam, lornoxicam, meloxicam, tenoxicam, COX-2 Inhibitors, such as, celecoxib, etoricoxib, lumiracoxib, parecoxib, rofecoxib, valdecoxib; and Sulphonanilides, such as nimesulide.

Glucocorticoids influence all types of inflammatory events. For example, the two main products in inflammation, Prostaglandins and Leukotrienes are inhibited by the action of Glucocorticoids. Glucocorticoids include, for example, Aldosterone, Cortisone, Hydrocortisone/cortisol, Desoxycortone, Alclometasone, Amcinonide, Beclometasone, Betamethasone, Budesonide, Ciclesonide, Clobetasol, Clobetasone, Clocortolone, Cloprednol, Cortivazol, Deflazacort, Deoxycorticosterone, Desonide, Desoximetasone, Dexamethasone, Diflorasone, Diflucortolone, Difluprednate, Fluclorolone, Fludrocortisone, Fludroxycortide, Flumetasone, Flunisolide, Fluocinolone acetonide, Fluocinonide, Fluocortin, Fluocortolone, Fluorometholone, Fluperolone, Fluprednidene, Fluticasone, Formocortal, Halcinonide, Halometasone, Hydrocortisone aceponate, Hydrocortisone buteprate, Hydrocortisone butyrate, Loteprednol, Medrysone, Meprednisone, Methylprednisolone, Methylprednisolone aceponate, Mometasone furoate, Paramethasone, Prednicarbate, Prednisone, Prednisolone, Prednylidene, Rimexolone, Tixocortol, Triamcinolone, and Ulobetasol.

On occasion, an intrathecally-implantable depot contains one or more analgesic compounds selected from a COX-2 inhibitor, morphine, hydromorphone, and biphalin. On other occasions, an intrathecally-implantable depot contains one or more analgesic compounds selected from morphine sulfate, ziconotide, bupivacaine, clonidine, and ketamine.

Pain-relieving drugs of the present disclosure generally do not include general anesthetic compounds. General anesthetic compounds cause lack of feeling or awareness, typically used to depresses the central nervous system reversibly, producing loss of consciousness, and muscle relaxation, with minimal depression of a patient's vital functions. General anesthetics include hydrocarbons, such as cyclopropane and ethylene, halogenated hydrocarbons, such as chloroform and trichloroethylene; ethers; and other compounds, such as tribromoethanol, nitrous oxide, and barbituates.

On occasion, an intrathecally-implantable depot includes a pain-relieving drug in combination with one or more additional medications for treatment of neuropathic pain. On occasion, an intrathecally-implantable depot includes a pain-relieving drug in combination with one or more antispastic drugs, one or more anti-depressant drugs, or combinations thereof. Antispastic drugs include, but are not limited to baclofen, dantolene, diazepam, and gabapentin. Anti-depressant drugs include, but are not limited to, selective serotonin reuptake inhibitors (SSRIs), serotonin-norepinephrine reuptake inhibitors (SNRIs), noradrenergic and specific serotonergic antidepressants (NASSAs), norepinephrine (noradrenaline) reuptake inhibitors (NRIs), norepinephrine-dopamine reuptake inhibitors, tricyclic antidepressants (TCAs), and monoamine oxidase inhibitor (MAOIs).

### Polymers

Polymers used in the intrathecally-implantable depot are typically degradable in vivo, i.e. biodegradable in the intrathecal space. Generally, a biodegradable polymer includes those polymers, including co-polymers and polymer blends, which degrade in vivo wherein at least 50% of the polymer degrades into non-toxic residues which are removed by the body within a year. For example, biodegradable polymers degrade in vivo by methods such as hydrolysis, bulk erosion, or surface erosion.

A biodegradable polymer is typically selected for use in portions of the intrathecally-implantable depot, i.e., biodegradable matrices or biodegradable casings, based upon the desired time release profile of pain-relieving drug associated therewith. Biodegradable polymers having different drug release profiles, which relate to their rate and method of degradation, are available. In addition, to biodegradability characteristics, biodegradable polymers are on occassion selected to impart flexibility to intrathecally-implantable depot, or portions thereof.

Generally, at least one biodegradable polymer for use in degradable matrix or degradable casing of the depot degrades slowly, with at least 50% of a biodegradable polymer remaining at six months. In other instances, a biodegradable polymer for use in degradable matrix or degradable casing of the depot degrades slowly, with at least 50% of a biodegradable polymer remaining at nine months. Occasionally, a slow-degrading polymer for use in degradable matrix or degradable casing degrades slowly, with 50% of a biodegradable polymer remaining at a year.

In some instances, the intrathecally-implantable depot, in addition to a slow-degrading polymer, also includes at least one biodegradable polymer which significantly degrades within a month. Typically, a fast-degrading polymer will have at least 50% of the polymer degraded within a month and complete release of the pain-relieving drug held by the fast-degrading polymer within a two week period.

Biodegradable polymer or polymers suitable for use in the intrathecally-implantable depot typically include, but are not limited to: polyesters, polyorthoesters, polyphosphoesters, polycarbonates, polyanhydrides, polyphosphazenes, polyoxalates, polyaminoacids, polyhydroxyalkanoates, polyethyleneglycol, polyvinylacetate, copolymers and blends thereof, and the like (see U.S. Pat. No. 4,675,189; U.S. Pat. No. 4,767,628; U.S. Pat. No. 5,271,945; U.S. Pat. No. 5,618,563; U.S. Pat. No. 7,205,378; WO 93/20126; GB Patent No. 2,145,422). In some instances, biodegradable polymer or polymers suitable for use in the intrathecally-implantable depot include polyhydroxyacids, polyanhydrides, or combinations thereof. On occasion, the intrathecally-implantable depot includes co-polymers of lactic and glycolic acid or combinations thereof. In some instances, suitable co-polymers of lactic and glycolic acid have a monomer ratio selected from the range of 50:50 to 90:10. On other occasions, the intrathecally-implantable depot includes ethylene-vinyl acetate co-polymers, often containing 20-50 wt. % vinyl acetate, for example 40 wt. % vinyl acetate.

In various embodiments, the depot has at least one biodegradable casing or biodegradable matrix selected from a copolymer of lactic acid and glycolic acid at a ratio of 85:15 (PLGA-85:15), a copolymer of lactic acid and glycolic acid at a ratio of 75:25 (PLGA-75:25) or a copolymer of lactic acid and glycolic acid at a ratio of 50:50 (PLGA-50:50).

### III. FABRICATION

A intrathecally-implantable depot of the present disclosure may be generally fabricated by loading one or more biodegradable polymers with a pain-relieving drug, or composition thereof, and shaping as desired. A pain-relieving drug may be in a composition in the form of a liquid solution, powder, granules, pellets, tablets, capsules, and the like using pharmaceutically acceptable excipients and techniques.

Some intrathecally-implantable depots or portions (e.g., units) thereof are made of biodegradable polymers which release entrapped pain-relieving drug as the polymer is degraded within the body. In these depots, the rate of matrix or casing erosion (degradation in vivo) determines the rate and order in which the drug or drugs are released. Other intrathecally-implantable depots or portions (e.g., units) thereof are made of a high porosity matrix which relies on the time it takes a drug located within the pores of the matrix to diffuse from the intrathecally-implantable depot. Still other portions (e.g., units) of intrathecally-implantable depots are made of pain-relieving drug, or compositions thereof including pharmaceutically acceptable excipients, surrounded by biodegradable casing where the degradation of a biodegradable casing determines the rate and order in which the drug or drugs are released.

Generally, the intrathecally-implantable depots of the present disclosure have a high pain-relieving drug loading. Typically, the pain-relieving drug or composition thereof is at least 50% w/w of the intrathecally-implantable depot. Frequently, the pain-relieving drug or composition thereof is at least 70% w/w of the intrathecally-implantable depot. The pain-relieving drug or composition thereof may on occasion be at least 90% w/w of the intrathecally-implantable depot.

One or more pain-relieving drugs may be coated with a biodegradable polymer, thereby forming a biodegradable polymer unit having a biodegradable casing. One or more pain-relieving drugs may be embedded into a biodegradable polymer matrix thereby forming a biodegradable polymer unit having a biodegradable matrix. On occasion, the biodegradable polymer matrix is flexible.

One or more of the biodegradable polymer units may be optionally assembled together, for example with an additional, same or different, biodegradable polymer. Additionally, individual loaded biodegradable polymer units and/or an assembly of multiple biodegradable polymer units may be optionally coated an additional, same or different, biodegradable polymer to form a biodegradable casing thereon. On occasion, the biodegradable casing is flexible.

In some methods, biodegradable polymers and drug mixtures are blended and/or formed into granulates or other particles, wherein the drug-polymer granulation is packed into a casing of an intrathecally-implantable depot or portion or unit thereof.

In some other methods, biodegradable polymers and drug mixtures are blended and formed, typically by compression into units or other portions of an intrathecally-implantable depot. Optionally, the units or other portions are coated by spraying or dipping or rolling in the casing polymer prior to assembly. In a further option, the units or other portions are assembled and are coated by spraying or dipping or rolling in the casing polymer.

Extrusion techniques are also applicable to preparation of an intrathecally-implantable depot of the present disclosure. In some processes, biodegradable polymers and drug mixtures are blended and co-extruded. On occasion, extrusion processes are employed to prepare intrathecally-inplantable depot, including biodegradable cores, units or portions thereof.

The pain-relieving drug is “dispersed” or “loaded” within the biodegradable matrix if the pain-relieving drug is directly or indirectly, physically or chemically held by the biodegradable matrix at a location other than, or in addition to, the surface of the matrix. Typically, the pain-relieving drug is held throughout the biodegradable matrix. Generally, a pain-relieving drug may be physically bound to a biodegradable matrix by dispersing, entrapping, imbedding or otherwise containing the pain-relieving drug within the matrix. Frequently, co solvation or coprecipitation techniques are used to achieve physical association between a pain-relieving drug and materials of the biodegradable matrix. On occasion, a pain-relieving drug may be chemically bound to the matrix by way of a chemical reaction wherein the pain-relieving drug is covalently or ionically bonded to a matrix material. Similarly, pain-relieving drug may be dispersed or loaded within a biodegradable coating. These and other techniques for associating the pain-relieving drug in the matrices or coatings of the depot are contemplated.

Processes for loading and shaping biodegradable polymer compositions incorporating pain-relieving drug or compositions thereof is generally performed according to known methods, e.g., methods described in U.S. Pat. Nos. 5,456,917 and 5,718,921, and/or as described below.

Frequently, pain-relieving drugs are loaded into biodegradable polymers by dry processing, but on occasion, pain-relieving drugs are loaded into biodegradable polymers by wet processing. For example, the pain-relieving drug or composition thereof is dissolved in a solvent such as acetic acid and freeze-dried prior to combining with the polymer composition. A biodegradable polymer in the form of an extrudate, fibrous precipitate, or a foam is ground and blended with the freeze-dried drug composition. Frequently, the biodegradable polymer and the pain-relieving drug composition are mixed as dry powders, but mixing by wet processing may alternatively be performed. The mixture is then extruded into desired shapes and forms under pressure and/or heat. For example, the extruding step is carried out under 20,000 tons of pressure. Alternatively, the extrusion is carried out at 40,000 tons of pressure and up to 100,000 tons of pressure. In some instances, the manufacturing process is typically carried out at a temperature at or below 37 degrees centigrade to avoid degradation of the pain-relieving drug or biodegradable polymer.

An alternative method of loading the pain-relieving drug into the polymer particles is by preparing a slurry of polymer particles in a solution of pain-relieving drug and applying a vacuum to fill the voids of the particles. This result may be accomplished by selectively dissolving the drug without dissolving or causing swelling or other morphological changes in the polymer. Preferably, the solvent should have a high vapor pressure so that it may easily be removed by volatilization or sublimation. The pain-relieving drug solution is then added to the foam, which itself may be ground to a particular particle size range, to form a slurry. In order to force the pain-relieving drug into the pore structure, the slurry is degassed, either as a liquid/solid mixture or after freezing. This process effectively removes air from the pores. After degassing, the slurry, if frozen, is thawed so that the drug solvent melts and air, or any other suitable gas, is allowed to enter the system at any desired pressure. The gas pressure forces the drug solution into the pores. The mixture is then re-frozen and the solvent is then removed by lyophilization, a process which traps the pain-relieving drug within the pores.

Intrathecally-implantable depots of the present disclosure may be fabricated in a variety of sizes and shapes, for example to accommodate differences in patient size and/or total drug load, which varies according to dosage and time of delivery. Generally, an intrathecally-implantable depot has a cross-sectional diameter no greater than 1500 microns (1.5 mm). Generally, an intrathecally-implantable depot has a cross-sectional diameter no less than 50 microns (0.05 mm). Typically, a depot has a cross-sectional diameter in an inclusive range from 50 microns to 1400 microns; often in an inclusive range from 100 microns to 1200 microns; an even on occasion in an inclusive range of 400 microns to 1200 microns. Typically, a depot is sized to fit within a needle, wherein the gauge of the needle is selected according to the Stubs needle gauge size range from between 15 gauge to 32 gauge inclusive. On occasion, a depot is sized to fit within an 18 gauge needle. Often depot has a cross-sectional diameter in an inclusive range between 0.08 mm to 1.37 mm inclusive. Depots having a cross-sectional diameter no greater than: 89, 114, 140, 165, 191, 241, 292, 318, 394, 495, 584, 686, 838, 1067, 1194, or 1372 microns, and all integers in between, are envisioned for depots suitable for intrathecal implantation. It is generally accepted that the size of the depot selected for use is at least in part based upon the size of the patient, for example, the estimated or actual size and shape of the intrathecal space.

Generally, the length of the depot greatly exceeds the maximum diameter and is sufficient, in combination with cross-sectional area, to hold the total amount pain-relieving drug for delivery. The length of the depot is generally no greater than 30 cm; frequently, no greater than 25 cm; often, no greater than 20 cm, occasionally, no greater than 15 cm; sometimes, no greater than 10 cm; other times, no greater than 5 cm, and even on occasion no greater than 3 cm. Generally, the length of the depot is at least 1 cm; typically the length of the depot is at least 1.5 cm; an on occasion at least 2 cm.

The length of the depot is generally in an inclusive range of 1 cm to 30 cm. Frequently, the length of the depot is in an inclusive range of 5 cm to 25 cm inclusive. Sometimes, the length of the depot is in an inclusive range from 1 to 10 cm. On occasion, the length of the depot is in an inclusive range from 1 cm to 5 cm. On still further occasions, the length of the depot is in an inclusive range from 1.5 cm to 3 cm. In some instances, the length of the depot relates to or is adjusted to hold the total amount pain-relieving drug to be delivered.

Generally, the depot releases pain-relieving drug at a rate of 0.1-25 mg/day. For example, pain-relieving drug is released at a rate of 3.5 mg/day. The degradation rate of the depot is such that full release of pain-relieving drug is achieved within a period of 1 month to 1 year. Often, degradation rate of the depot is such that full release of pain-relieving drug is achieved within a time release period of 1 month to 9 months, sometimes within a time release period of 1 month to 6 months, on occasion within a time release period of 1 month to 3 months, or even within a time release period of 1 month to 2 months. In some instances, release of drug over a time release period of 2-3 months is preferred.

### IV. METHODS OF PAIN MANAGEMENT

The methods of long-term pain management using a intrathecally-implantable depot of the present disclosure generally include implantation of the depot into the intrathecal space of the spine of a patient in need of relief from pain. Typically, the pain is visceral pain and/or neuropathic pain. Typically, implantation of an intrathecally-implantable depot of the present disclosure into the intrathecal space of the spine of a patient provides a baseline level of pain relief. In addition, a patient Frequently, pain in a patient is assessed on a scale of 0-10. A representative scale is
0 No pain.
1-2 Annoying to patient, but bearable; patient may seek a remedy or may ignore the pain.
3-4 Sufficiently painful to cause patient to seek a remedy.
5-6 Interferes with patient's ability to focus on normal activities; stronger relief is needed.
7-8 Dealing with the pain has become patient's first priority; patient is prevented from doing normal activities. Pain is near to unbearable for patient.
9-10 The worst pain patient has ever experienced. The pain is unbearable for patient.

A baseline level of pain relief is typically considered pain relief for a patient to generally score less than 5 on a pain assessment. On occasion, a baseline level of pain relief is considered pain relief for a patient to score less than 3 on a pain assessment. Generally, a baseline level of pain relief in methods of the present disclosure is monitored over a period of time, e.g. treatment period, with the baseline level being an average of individual assessments over the treatment period. Occasionally, during long-term pain management, a patient will experience breakthrough pain, wherein the pain level increases above the desired baseline level. Typically, long-term pain management using an intrathecally-implantable depot of the present disclosure allows further administration of other additional pain medications for treatment of breakthrough pain. Additional administration includes, but is not limited to topical, enteral, and/or parenteral administration.

Patients in need of long-term relief from pain include those suffering from chronic pain, as well as those whose pain has continued or is likely to continue for a month or more. Generally, methods according to the disclosure provide long-term administration of a pain-relieving drug or combination of pain-relieving drugs, thereby managing or alleviating the patient's pain without making the patient unconscious.

Management of chronic pain via implantation of a intrathecally-implantable depot disclosed herein is useful for any condition including chronic pain enduring of a month or more. Example conditions including chronic pain which may be managed by implantation of a intrathecally-implantable depot described herein include: failed back surgery syndrome, reflex sympathetic dystrophy, causalgia, arachnoiditis, chronic pancreatitis, and cancer pain. In some instances, the cancer pain is pain related to metastatic cancer, pain related to tumors compressing the spinal nerves, and/or pain related to scarring from previous radiation therapy.

Frequently, the depots and methods of the present disclosure additionally limit, reduce or avoid the adverse side effects of pain-relieving drugs as compared to the adverse side effects of the same pain-relieving drugs administered systemically, such as toxicity, hypotension, sedation, nausea, vomiting, and constipation. Typically, smaller amounts of pain-relieving drugs are needed by the depots and methods of the present disclosure to alleviate the subject's pain as compared to systemic delivery of the same pain-relieving drugs over the same time period. Also, the depots and methods of the present disclosure have the capability to provide both rapid and prolonged pain management. In addition, diffuse pain, pain of unknown origin, or pain in multiple areas of the patient's body may be alleviated by the depots and methods of the present disclosure without requiring determination of the pain source, without requiring direct delivery of pain-relieving drug directly to the source of the pain, and without requiring systemic delivery. Furthermore, the methods and depots of the present disclosure have decreased the risk of tolerance development for pain management with opiates.

In an example method of the present disclosure, pain-relieving drug is administered by implantation of a intrathecally-implantable depot of the present disclosure into the intrathecal space. Generally, the intrathecally-implantable depot is placed into the intrathecal space and drug is released by diffusion and/or as a result of depot degradation over a time release period of a month or more. Typically, the intrathecally-implantable depot releases a therapeutically-effective amount of pain-relieving drug in a sustained manner, and in most instances a linear manner. Alternatively or additionally, the intrathecally-implantable depot releases pain-relieving drug in a plurality of discrete, sequential doses over a time release period. In some embodiments, the intrathecally-implantable depot additionally releases an additional, same or different, pain-relieving drug in an initial short-duration release of two-weeks or less following implantation. In select embodiments, the intrathecally-implantable depot additionally releases an additional, same or different, pain-relieving drug in an initial burst release following implantation.

### V. EXAMPLE

One specific embodiment of self-contained depot for extended release drug delivery of the present disclosure is a completely biodegradable system. Granules of an analgesic drug in ethylene-vinyl acetate copolymer (EVAc)(for example, 40 wt. % vinyl acetate) will be compressed into a flexible fiber having a maximum diameter of about 0.63 mm and a length of about 10 cm. The fiber will be coated with EVAc creating a biodegradable shell of 0.1 thickness along the length of the fiber. The self-contained depot will be a coated fiber having a final maximum diameter of less than or equal to 0.838 mm to fit within an 18 gauge needle.

All publications and patent applications in this specification are indicative of the level of ordinary skill in the art to which this disclosure pertains and are incorporated herein by reference in their entireties.

The various embodiments described above are provided by way of illustration only and should not be construed to limit the disclosure. Those skilled in the art will readily recognize various modifications and changes that may be made to the present disclosure without following the example embodiments and applications illustrated and described herein, and without departing from the true spirit and scope of the present invention.

The claimed invention is:

1. A self-contained depot for extended release drug delivery comprising:
an intrathecally-implantable biodegradable core; and
analgesic composition is secured within the biodegradable core;
wherein the biodegradable core and analgesic composition are together configured to have a time release period of at least one month.

2. The self-contained depot of claim 1, wherein said depot is in the shape of a rod having a diameter no greater than 1.5 mm.

3. The self-contained depot of claim 1, wherein said biodegradable core is flexible.

4. The self-contained depot of claim 1 additionally comprising an outer casing surrounding the biodegradable core.

5. The self-contained depot of claim 4, wherein said outer casing is a non-biodegradable polymer and has at least one opening allowing fluid communication between the biodegradable core and the spinal fluid in the intrathecal space.

6. The self-contained depot of claim 1, wherein said analgesic compounds comprise at least 50% w/w of said depot.

7. The self-contained depot of claim 1, wherein said biodegradable core comprises polymer materials selected from the group consisting of polyanhydride polymer, poly lactic-glycolic acid (PLGA) polymer, or combinations thereof.

8. A method for preparing a self-contained intrathecally-implantable depot comprising:
embedding an analgesic composition within a biodegradable material;
forming said biodegradable material into a biodegradable core, wherein said biodegradable core is configured to biodegrade at least 50% over a time period of one to 12 months.

9. A method of continuously relieving pain in a subject comprising:
implanting into said subject an self-contained depot comprising a intrathecally-implantable biodegradable core, and analgesic composition held in the biodegradable core;
biodegrading said biodegradable core in vivo over a time period of at least one month; and
releasing a therapeutically-effective amount of analgesic composition from the biodegrading core over a time period of at least one month.

10. An implant comprising
a plurality of units bonded together;
an analgesic composition disposed in each unit in an extended-release configuration; and
wherein the plurality of units together is configured for in vivo biodegradability over a time period of at least one month.

11. The implant of claim 10, wherein the plurality of units bonded together is in the form of a flexible rod.

12. The implant of claims 11, wherein the plurality of units bonded together has a diameter to fit in a 15-32 gauge needle.

13. The implant of claim 12, wherein each unit is a longitudinal section of the plurality of units bonded together or wherein each unit is a cross-sectional portion of the plurality of units bonded together.

14. The implant of claim 13, wherein a different analgesic composition is disposed within two or more units.

15. The implant of claims 14, wherein at least one unit degrades over a time period greater than one month and at least one other unit degrades over a time period of 14 days or less.

16. The implant of claim 13, comprising 2-4 units, 5-20 units, 21-100 units, or in excess of 100 units.

17. The implant of claim 16 additionally comprising an outer casing surrounding the plurality of units bonded together.

18. A flexible fiber for extended-release delivery of an analgesic, the fiber comprising an analgesic composition in a biodegradable core having a diameter of no greater than 1.5 mm, the biodegradable core provided by a step of encasing said analgesic composition in biodegradable materials having a characteristic of being at least 50% biodegradable over a time period of one to 12 months, and forming said biodegradable materials into a biodegradable core.

19. The implant of claim 10, wherein the units have a uniform size.

20. The implant of claim 10, wherein the units have a plurality of different sizes.
